(19) United States
(12) Patent Application Publication
(10) Pub. No.: US 2024/0307426 A1
(43) Pub. Date: Sep. 19, 2024

(54) USE OF SGLT-2 INHIBITORS FOR THE PREVENTION AND/OR TREATMENT OF RENAL DISEASES IN NON-HUMAN MAMMALS

(71) Applicant: BOEHRINGER INGELHEIM VETMEDICA GMBH, Ingelheim am Rhein (DE)

(72) Inventors: Carla KROH, Oestrich-Winkel (DE); Ingo Ulrich LANG, Ingelheim am Rhein (DE); José MATALLO, Frankfurt am Main (DE)

(21) Appl. No.: 18/578,207

(22) PCT Filed: Jul. 26, 2022
(86) PCT No.: PCT/EP2022/070943

§ 371 Date: Jan. 10, 2024

(30) Foreign Application Priority Data
Jul. 28, 2021 (EP) 21188321.0

(51) Int. Cl. A61K 31/7056; A61K 31/382; A61K 31/7034; A61K 31/7048; A61P 13/12

(52) U.S. Cl. CPC A61K 31/7056; A61K 31/382; A61K 31/7034; A61K 31/7048; A61P 13/12

(57) ABSTRACT

The present invention is directed to the use of one or more SGLT-2 inhibitors or pharmaceutically acceptable forms thereof for the prophylaxis and/or treatment of one or more renal diseases in a non-human mammal, such as a carnivore, in particular a cat or a dog.

### FIELD OF THE INVENTION

The invention relates to the field of medicine, in particular to the field of veterinary medicine. The invention relates to the use of one or more SGLT-2 inhibitors or a pharmaceutically acceptable form thereof in the prevention and/or treatment of one or more renal diseases in a non-human mammal, such as a carnivore, in particular a cat or a dog.

### BACKGROUND INFORMATION

Chronic kidney disease (CKD) is a major cause of morbidity and mortality in companion animals, especially in carnivores as the cat and the dog, with a prevalence of 1.0-3.0% in cats and 0.5-1.0% in dogs, increasing substantially in old age. Nephron damage associated with CKD is usually irreversible and often progressive.

The international renal interest society (IRiS) categorized CKD in different stages based initially on fasting blood creatinine or fasting blood symmetric dimethylarginine (SDMA) concentration or (preferably) both assessed on at least two occasions in a hydrated, stable patient. The patient is then sub-staged based on proteinuria and blood pressure into the following stages:
1—Normal blood creatinine or normal or mild increase in blood SDMA. Some other renal abnormality present (abnormal renal palpation, or renal imaging findings, proteinuria of renal origin, abnormal renal biopsy results, increasing blood creatinine or SDMA concentrations in samples collected serially). Persistently elevated blood SDMA concentration (>14 μg/dl) may be used to diagnose early CKD.
2—Normal or mildly increased creatinine, mild renal azotaemia (lower end of the range lies within reference ranges for creatinine for many laboratories, but the insensitivity of creatinine concentration as a screening test means that patients with creatinine values close to the upper reference limit often have excretory failure). Mildly increased SDMA. Clinical signs usually mild or absent.
3—Moderate renal azotemia. Many extrarenal signs may be present, but their extent and severity may vary. If signs are absent, the case could be considered as early Stage 3, while presence of many or marked systemic signs might justify classification as late Stage 3.
4—Increasing risk of systemic clinical signs and uremic crises.

Therapeutic standard of care usually consists of ACE inhibitors (e.g. enalapril), calcium channel blockers (e.g. diltiazem and amlodipine), angiotensin receptor blocker (e.g. telmisartan), targeting renal haemodynamics. In addition, clinical signs are treated as needed (e.g. vomiting, nausea, body weight loss, inappetence) and renal diets are available for the management of the disease.

State of the art interventions are based on specific action on unique pathways for symptomatic treatment of secondary conditions induced by renal conditions. In humans, beneficial renal effects under the use of SGLT-2 inhibitors was observed (DAPA-CKD ClinicalTrials.gov number, NCT03036150). However, the direct effect on kidney of carnivores like felines and canines remains unknown. Noteworthy, there are anatomical, physiological and pathophysiological differences between the species. A first evidence for such differences becomes apparent by comparing the prevalence of CKD in cats compared to dogs (1.0-3.0% in cats and 0.5-1.0% in dogs) and humans (between 11 to 13% with the majority stage 3). Also, in contrast to people and dogs, primary glomerulopathies with marked proteinuria are remarkably rare findings in cats and the majority of geriatric cats with CKD do not have histologic evidence of primary glomerular disease. Typical histologic features include interstitial inflammation, tubular atrophy, and fibrosis with secondary glomerulosclerosis. In addition, in people, cardiovascular disease (CVD) is the primary cause of morbidity and mortality where CKD is regarded as an accelerator of CVD risk and an independent risk factor for CVD events. There is a graded inverse relationship between CVD risk and glomerular filtration rate (GFR) that is independent of age, sex and other risk factors. This is not the case in non-human mammals, e.g. carnivores, such as dogs and cats, and the pathogenesis of cardiovascular diseases (cardiomyopathies, valve diseases) differs significantly compared to the pathogenesis observed in people (coronary disease).

Wheeler et al. (Diabetes Ther (2020) 11:2757-2774) reviews the actions of SGLT-2 inhibitors in chronic kidney disease progression in type 2 diabetes human patients.

Dekkers and Gansevoort (Nephrol Dial Transplant (2020) 35: 133-142) describes the possible application of SGLT-2 inhibitors in non-diabetic kidney disease in humans.

EP 3 508 222 discloses an agent or pharmaceutical composition for eliminating senescent cells, comprising an SGLT2-inhibitor.

WO 2021/092341 discloses sodium-glucose linked transporter inhibitors for the management of chronic kidney disease, hypertension and heart failure in companion animals.

Notwithstanding the disclosures of the above documents, there is a medical need for the prevention and/or treatment of one or more renal diseases in a non-human mammal, such as a carnivore, in particular a cat or a dog.

### SUMMARY OF THE INVENTION

The present invention concerns one or more SGLT-2 inhibitors or pharmaceutically acceptable forms thereof for use in a method of prevention and/or treatment of one or more renal diseases in a non-human mammal, such as a carnivore, in particular a cat or a dog.

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as well as the corresponding use of one or more SGLT-2 inhibitors for the preparation of a medicament for the prevention and/or treatment of one or more renal diseases in a non-human mammal, such as a carnivore, in particular a cat or a dog, are also intended to be comprised by the present invention.

In one aspect, the present invention also concerns the one or more SGLT-2 inhibitors or pharmaceutically acceptable forms thereof for the uses as herein disclosed and/or claimed, wherein the one or more renal diseases are selected from the group consisting of: renal dysplasia, glomerulopathy, polycystic kidney disease, amyloidosis, tubulo-nephritis/tubulointerstitial nephritis (TIN), acute kidney disease, chronic kidney disease.

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In one aspect, the present invention also concerns the one or more SGLT-2 inhibitors or pharmaceutically acceptable forms thereof for the uses as herein disclosed and/or claimed, wherein the one or more renal diseases are selected from the group consisting of: acute kidney disease, chronic kidney disease.

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In one aspect, the present invention also concerns the one or more SGLT-2 inhibitors or pharmaceutically acceptable forms thereof for the uses as herein disclosed and/or claimed, wherein the one or more renal diseases are selected from the group consisting of: chronic kidney disease. A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In another aspect, the present invention also concerns the one or more SGLT-2 inhibitors or pharmaceutically acceptable forms thereof for the uses as herein disclosed and/or claimed, wherein the one or more SGLT-2 inhibitors are glucopyranosyl-substituted benzene derivatives.

A corresponding method of preventing and/or treating one or more renal diseases in a non-human mammal. such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors are selected from the group consisting of:
(1) a glucopyranosyl-substituted benzene derivative of the formula (1)

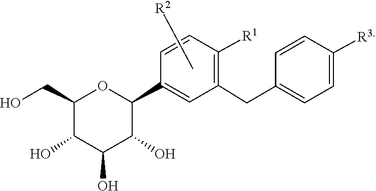

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoro-methyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetra-hydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, p henyl-carbonyl and phenyl-(C₁₋₃-alkyl)-carbonyl;
(2) Velagliflozin, represented by formula (2):

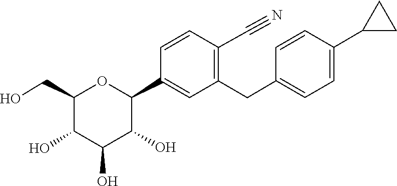 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

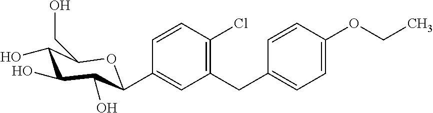

(4) Canagliflozin, represented by formula (4):

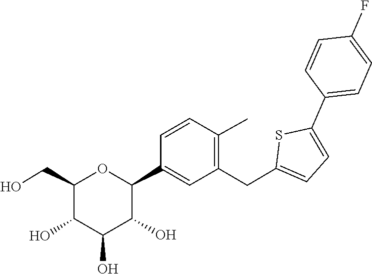 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

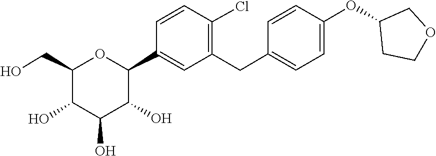 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luseogliflozin, represented by formula (6):

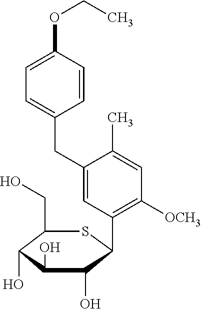

(7) Tofogliflozin, represented by formula (7):

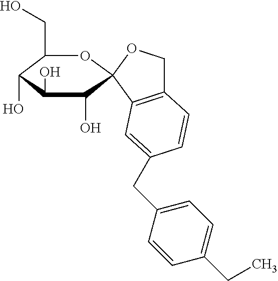

(8) Ipragliflozin, represented by formula (8):

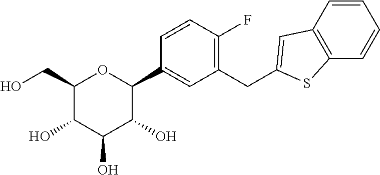 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

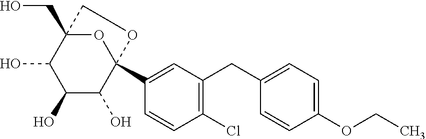 [SMILES: CCOc1ccc(Cc2cc([C@]34OC[C@](CO)(O3)[C@@H](O)[C@H](O)[C@H]4O)ccc2Cl)cc1]

(10) Atigliflozin, represented by formula (10):

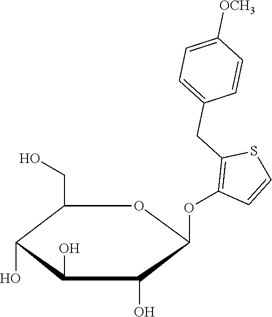

(11) Remogliflozin, represented by formula (11):

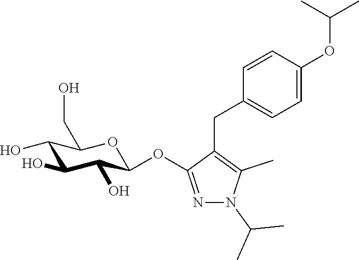

(11A) Remogliflozin ctabonate, represented by formula (11A):

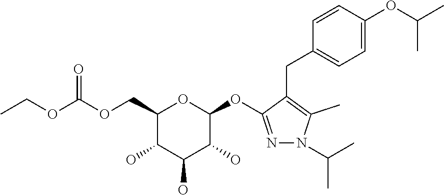 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

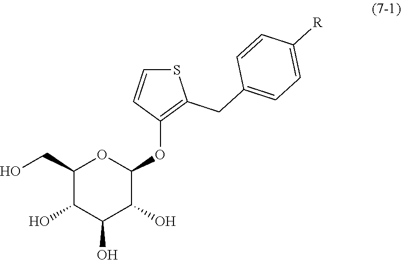

wherein R denotes methoxy or trifluoromethoxy;
(13) 1-(β-D-glucopyranosyl)-4-methyl-3-[5-(4-fluorophenyl)-2-thienylmethyl]benzene, represented by formula (13);

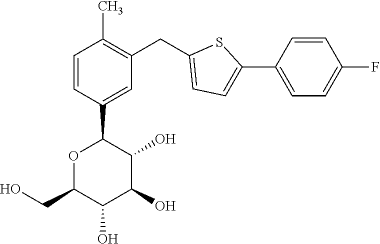 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

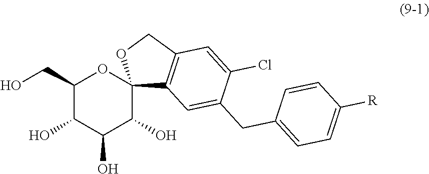

wherein R denotes methoxy, trifluoromethoxy, ethoxy, ethyl, isopropyl or tert, butyl;
(15) a pyrazole-O-glucoside derivative of the formula (15)

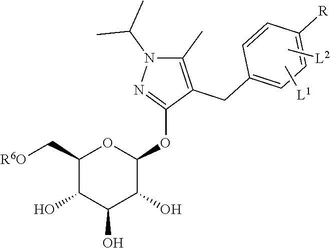

wherein
R¹ denotes C₁₋₃-alkoxy,
L¹, L² independently of each other denote H or F,
R⁶ denotes H, (C₁₋₃-alkyl)carbonyl, (C₁₋₆-alkyl)oxycarbonyl, phenyloxycarbonyl, benzyloxycarbonyl or benzylcarbonyl;
(16) Sotagliflozin, represented by formula (16):

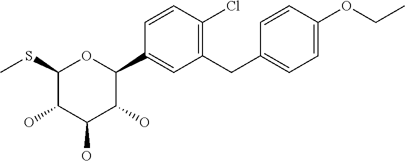 [SMILES: CCOc1ccc(Cc2cc([C@@H]3O[C@H](SC)[C@@H](O)[C@H](O)[C@H]3O)ccc2Cl)cc1]

(17) Sergliflozin, represented by formula (17):

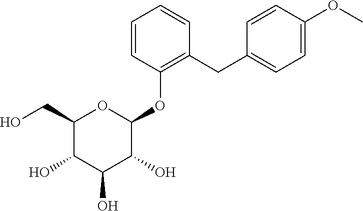 [SMILES: COc1ccc(Cc2ccccc2O[C@@H]2O[C@H](CO)[C@@H](O)[C@H](O)[C@H]2O)cc1]

(18) a compound represented by formula (18):

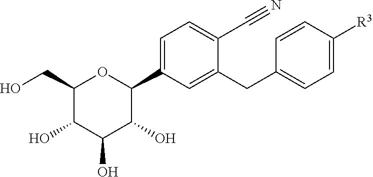

wherein
   R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

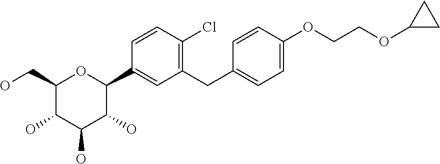 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

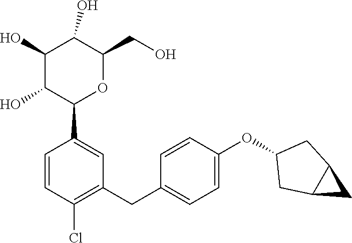 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin, represented by formula (21):

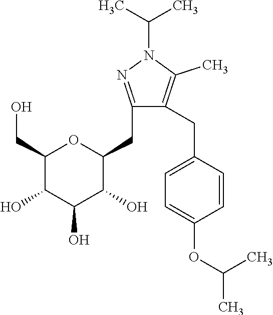 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin.

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the pharmaceutically acceptable form thereof is a crystalline complex between the one or more SGLT2 inhibitors and one or more amino acids, preferably proline, more preferably L-proline; and most preferably is co-crystal of the one or more SGLT2 inhibitors, L-proline and crystalline water.

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the non-human mammal, such as a carnivore, in particular a cat or a dog, is a non-human mammal patient in need of such prevention and/or treatment; preferably a carnivore patient in need of such prevention and/or treatment; more preferably is a cat patient or a dog patient in need of such prevention and/or treatment, even more preferably a non-diabetic cat patient or a non-diabetic dog patient in need of such prevention and/or treatment.

A corresponding method of preventing and/or treating one or more renal diseases in a non-human mammal, such as a carnivore, in particular a cat or a dog, comprising administering one or more SGLT-2 inhibitors to such non-human animal, preferably carnivore, in particular a cat or a dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors are administered orally, parenterally, intravenously, subcutaneously or intramuscularly, preferably orally.

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors are to be administered at a dose of 0.01 mg/kg bodyweight to 10 mg/kg bodyweight, preferably at a dose of 0.01 mg/kg bodyweight to 5 mg/kg bodyweight, more preferably at a dose of 0.01 mg/kg bodyweight to 4 mg/kg bodyweight, even more preferably at a dose of 0.01 mg/kg bodyweight to 3 mg/kg bodyweight, even more preferably at a dose of 0.01 mg/kg bodyweight to 2 mg/kg bodyweight, even more preferably at a dose of 0.01 mg/kg bodyweight to 1 mg/kg bodyweight, even more preferably at a dose of 0.01 mg/kg bodyweight to 0.5 mg/kg bodyweight, even more preferably at a dose of 0.01 mg/kg bodyweight to 0.3 mg/kg bodyweight, most preferably at a dose of 0.05 mg/kg bodyweight (dogs) or 1.0 mg/kg bodyweight (cats).

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein such one or more SGLT2 inhibitors or pharmaceutically acceptable forms thereof is to be administered once or twice per day. A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors is velagliflozin, which is to be administered as single SGLT-2 inhibitor, preferably orally. more preferably once or twice per day at a dose of 0.01 mg/kg bodyweight to 1 mg/kg bodyweight, even more preferably at a dose of 0.01 mg/kg bodyweight to 0.5 mg/kg bodyweight, even more preferably at a dose of 0.01 mg/kg bodyweight to 0.3 mg/kg bodyweight, most preferably at a dose of 0.05 mg/kg bodyweight (dogs) or 1.0 mg/kg bodyweight (cats).

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein velagliflozin as single SGLT-2 inhibitor is to be orally administered once daily at a dose of 0.01 mg/kg bodyweight to 1.0 mg/kg bodyweight, preferably once daily at a dose of 0.05 mg/kg bodyweight (dogs) or 1.0 mg/kg bodyweight (cats).

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the one or more SGLT-2 inhibitors are to be administered before, after or concomitantly with administering one or more other active pharmaceutical ingredients, preferably selected from the group consisting of: another SGLT-2 inhibitor or a pharmaceutically acceptable form thereof: one or more ACE inhibitors, such as benazepril, ramipril or enalapril; one or more calcium channel blockers, such as diltiazem or amlodipine; one or more angiotensin receptor blockers, such as telmisartan; one or more calcium-channel sensitizers and/or positive inotropes, such as pimobendan and/or digitalis alkaloids; and/or one or more phosphate binders, such as chitosan.

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

In yet another aspect, the present invention also concerns the one or more SGLT-2 inhibitors or pharmaceutically acceptable forms thereof for the uses as herein disclosed and/or claimed, wherein the preventive and/or therapeutic effect is characterized by one or more of the following clinical and/or biochemical parameters:
improved renal efficiency, characterized by a reduction of proteinuria—as well as a reduction and/or stabilization of serum SDMA and/or serum creatinine;
increase of the production of ketone bodies in the liver, characterized by increased plasma levels of 3-hydroxybutyric acid and/or the corresponding acylcarnitines i.e. hydroxy butyrylcarnitine and increased plasma levels of one or more of the branched-chain amino acids (e.g. valine, leucine and isoleucine);
improved blood pressure;
improved hydration status;
delayed onset of renal failure, preferably at least by 1, 2, 3, 4, 5, 6, 7, 8, 9, 10, 11, 12 or more months, or delayed and/or stopped progression of the one or more renal diseases, in particular chronic kidney disease, and/or improvement of the classification stage of the one or more renal diseases, in particular CKD (e.g. from stage III to stage II);
longer survival time of the non-human mammal patient, preferably at least by 1, 2, 3, 4, 5, 6, 7, 8, 9, 10, 11, 12 or more months and/or lower renal related mortality and/or morbidity;
improved clinical signs, such as reduced polydipsia, polyuria, vomiting and/or lethargy;
higher quality of life.

A corresponding method of preventing and/or treating one or more renal diseases in a non-human mammal, such as a carnivore, in particular a cat or a dog, comprising administering one or more SGLT-2 inhibitors to such non-human mammal, preferably carnivore, in particular cat or dog, as herein disclosed and/or claimed, as well as the corresponding use of one or more SGLT-2 inhibitors for the preparation of a medicament for the prevention and/or treatment of one or more renal diseases in a non-human mammal, such as a carnivore, in particular a cat or a dog, as herein disclosed and/or claimed, are also intended to be comprised by the present invention.

The present invention further concerns a pharmaceutical composition comprising one or more SGLT2 inhibitors or pharmaceutically acceptable forms thereof as herein disclosed and/or claimed for the uses/methods as herein disclosed and/or claimed.

The advantages according to the present invention are one or more of the following:
improved renal efficiency, characterized by a reduction of proteinuria—as well as a reduction and/or stabilization of serum SDMA and/or serum creatinine;
increase of the production of ketone bodies in the liver, characterized by increased plasma levels of 3-hydroxybutyric acid and/or the corresponding acylcarnitines i.e. hydroxybutyrylcarnitine and increased plasma levels of one or more of the branched-chain amino acids (e.g. valine, leucine and isoleucine);
improved blood pressure;
improved hydration status;
delayed onset of renal failure, preferably at least by 1, 2, 3, 4, 5, 6, 7, 8, 9, 10, 11, 12 or more months, or delayed and/or stopped progression of the one or more renal diseases, in particular chronic kidney disease, and/or improvement of the classification stage of the one or more renal diseases, in particular CKD (e.g. from stage III to stage II);
longer survival time of the non-human mammal patient, preferably at least by 1, 2, 3, 4, 5, 6, 7, 8, 9, 10, 11, 12 or more months and/or lower renal related mortality and/or morbidity;
improved clinical signs, such as reduced polydipsia, polyuria, vomiting and/or lethargy;
higher quality of life.

### DETAILED DESCRIPTION OF THE INVENTION

Before the embodiments of the present invention are described in further detail, it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

In the course of the present invention, the terms “carnivore” and “predominantly carnivorous non-human mammal” are used interchangeably. In one preferred embodiment the carnivore is a predominantly carnivorous non-human mammal, more preferred a canine, in particular a dog, and/or a feline, in particular a cat. In another preferred embodiment, the “non-human mammal” is selected from the group consisting of: bovine, canine, caprine, equine, feline, lagomorphs, ovine, porcine, rodent; more preferably is selected from the group consisting of: cattle, cow, dog, goat, horse, pony, donkey, cat, sheep, pig, rabbit, rat, mouse; even more preferably selected from the group consisting of: canine and/or feline; most preferably selected from the group consisting of: dog and/or cat.

Mammals are a class of vertebrate animals, whose females are characterized by the possession of mammary glands while both males and females are characterized by sweat glands, hair, three middle ear bones used in hearing, and a neocortex region in the brain. Within this class the placentals are preferred, which are characterized by the use of a placenta during gestation. Mammals can further be divided with respect to their feeding. Some mammals feed on animal prey-this is a carnivorous diet (and includes insectivorous diets). Other mammals, called herbivores, eat plants. An omnivore eats both prey and plants. Carnivorous mammals have a simple digestive tract, because the proteins, lipids, and minerals found in meat require little in the way of specialized digestion. Plants, on the other hand, contain complex carbohydrates, such as cellulose. The digestive tract of an herbivore is therefore host to bacteria that ferment these substances and make them available for digestion. The present invention is especially designed for carnivores and predominantly carnivorous non-human mammals. Such mammals include especially all feliforms, such as domestic cats or big cats, and most caniforms, such as the dogs, wolves and foxes. Due to the economic importance of companion animals in modern life, the present invention is especially designed for dogs and/or for cats.

In the course of the present invention, the term “renal dysplasia” refers to a rare renal malformation in which the kidney(s) are present but their development is abnormal, leading to malformation of histologic architecture of the kidney.

In the course of the present invention, the term “glomerulopathy” refers to the occurrence of glomerulonephritis with or without an accompanying condition.

In the course of the present invention, the term “polycystic kidney disease” refers to an inherited disorder in which liquid-filled sacs develop in the tissue of the kidney.

In the course of the present invention, the term “amyloidosis” refers to a group of diseases in which abnormal proteins, known as amyloid fibrils, build up in (kidney) tissue.

In the course of the present invention, the term “acute kidney disease” refers to a sudden episode of kidney failure or kidney damage.

In the course of the present invention, the terms “tubulo-nephritis” and “tubulointerstitial nephritis (TIN)” are used interchangeably and refer to a frequent cause of acute kidney injury (AKI) that can lead to chronic kidney disease (CKD). TIN is associated with an immune-mediated infiltration of the kidney interstitium by inflammatory cells, which may progress to fibrosis.

In the course of the present invention, the term “chronic kidney disease (CKD)” refers to the pathophysiological process, in which the remaining renal function is not enough to maintain the glomerular filtration rate at physiologic levels. It can also be defined as a complex clinical syndrome, that is based on abnormal structure and function of the kidneys and is characterized by weight loss, halitosis, poor hair quality, proteinuria, high blood pressure, azotaemia, polyuria, polydipsia, vomiting and anaemia. It is mostly a chronic condition, due to a progression in nephron damage, initiated by different factors, including diabetes, ischemic insults, toxic insults, some viral infections and cardiac conditions leading to high blood pressure.

SGLT-2 inhibitors for use according to the invention include, but are not limited to, glucopyranosyl-substituted benzene derivatives, for example as described in WO 01/27128, WO 03/099836, WO 2005/092877, WO 2006/034489, WO 2006/064033, WO 2006/117359, WO 2006/117360, WO 2007/025943, WO 2007/028814, WO 2007/031548, WO 2007/093610, WO 2007/128749, WO 2008/049923, WO 2008/055870, WO 2008/055940, WO 2009/022020 or WO 2009/022008.

Moreover, the one or more SGLT-2 inhibitors for use according to the invention may be selected from the group consisting of the following compounds or pharmaceutically acceptable forms thereof:
(1) a glucopyranosyl-substituted benzene derivative of the formula (1)

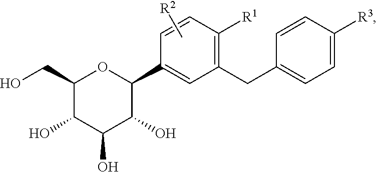

wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoro-methyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetra-hydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenyl-carbonyl and phenyl-(C₁₋₃-alkyl)-carbonyl;
(2) Velagliflozin, represented by formula (2):

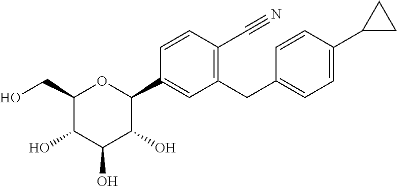 [SMILES: N#Cc1ccc([C@@H]2O[C@H](CO)[C@@H](O)[C@H](O)[C@H]2O)cc1Cc1ccc(C2CC2)cc1]

(3) Dapagliflozin, represented by formula (3):

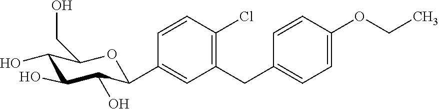

(4) Canagliflozin, represented by formula (4):

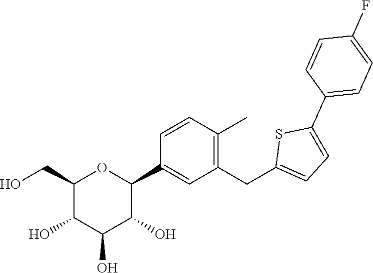 [SMILES: Cc1ccc([C@@H]2O[C@H](CO)[C@@H](O)[C@H](O)[C@H]2O)cc1Cc1ccc(-c2ccc(F)cc2)s1]

(5) Empagliflozin, represented by formula (5):

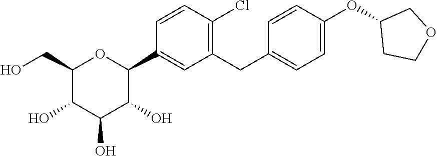 [SMILES: OC[C@H]1O[C@@H](c2ccc(Cl)c(Cc3ccc(O[C@H]4CCOC4)cc3)c2)[C@H](O)[C@@H](O)[C@@H]1O]

(6) Luscogliflozin, represented by formula (6):

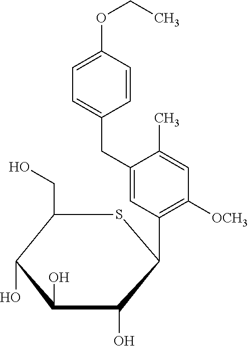

(7) Tofogliflozin, represented by formula (7):

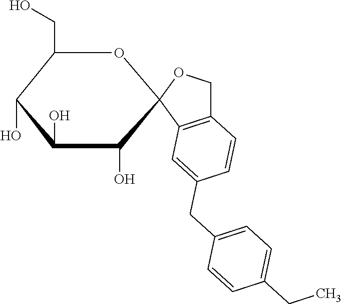

(8) Ipragliflozin, represented by formula (8):

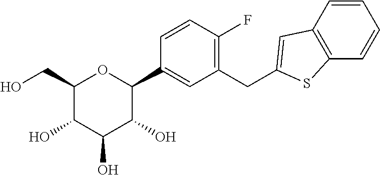 [SMILES: OC[C@H]1O[C@@H](c2ccc(F)c(Cc3cc4ccccc4s3)c2)[C@H](O)[C@@H](O)[C@@H]1O]

(9) Ertugliflozin, represented by formula (9):

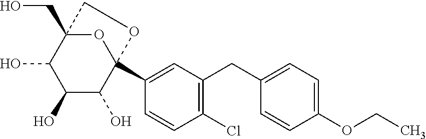

(10) Atigliflozin, represented by formula (10):

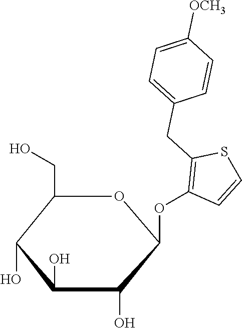

(11) Remogliflozin, represented by formnia (11):

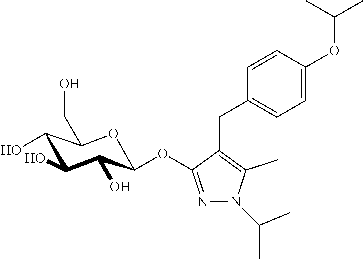

(11A) Remogliflozin ctabonate, represented by formula (11A):

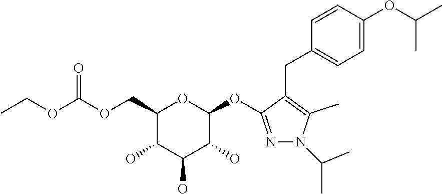 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

(12) a thiophene derivative of the formula (12)

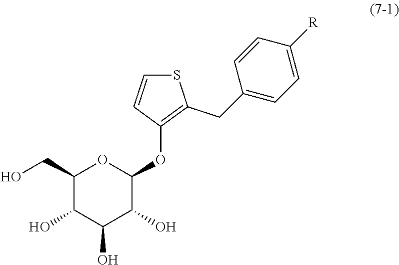

wherein R denotes methoxy or trifluoromethoxy;
(13) 1-(β-D-glucopyranosyl)-4-methyl-3-[5-(4-fiorophenyl)-2-thienylmethyl]benzene, represented by formula (13);

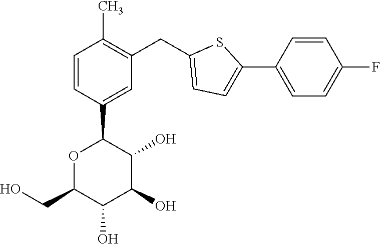 [SMILES: Cc1ccc([C@@H]2O[C@H](CO)[C@@H](O)[C@H](O)[C@H]2O)cc1Cc1ccc(-c2ccc(F)cc2)s1]

(14) a spiroketal derivative of the formula (14):

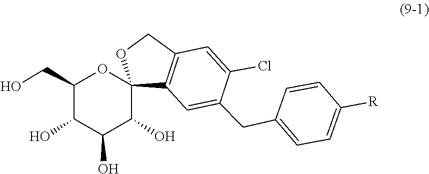

wherein R denotes methoxy, trifluoromethoxy, ethoxy, ethyl, isopropyl or tert. butyl;
(15) a pyrazole-O-glucoside derivative of the formula (15)

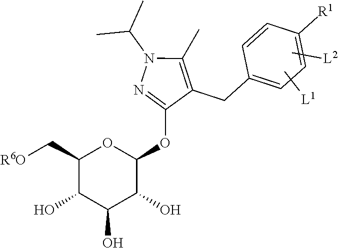

wherein
R¹ denotes C₁₋₃-alkoxy,
L¹, L² independently of each other denote H or F,
R⁶ denotes H, (C₁₋₃-alkyl)carbonyl, (C₁-6-alkyl)oxycarbonyl, phenyloxycarbonyl, benzyloxycarbonyl or benzylcarbonyl;
(16) Sotagliflozin, represented by formula (16):

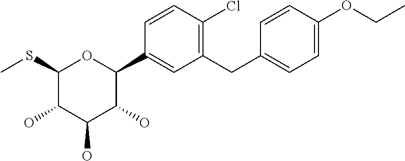 [SMILES: CCOc1ccc(Cc2cc([C@@H]3O[C@H](SC)[C@@H](O)[C@H](O)[C@H]3O)ccc2Cl)cc1]

(17) Sergliflozin, represented by formula (17):

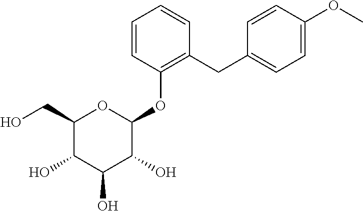 [SMILES: COc1ccc(Cc2ccccc2O[C@@H]2O[C@H](CO)[C@@H](O)[C@H](O)[C@H]2O)cc1]

(18) a compound represented by formula (18):

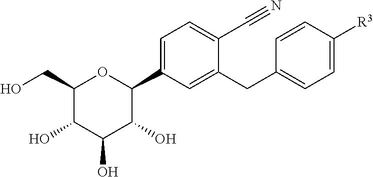

wherein
   R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19):

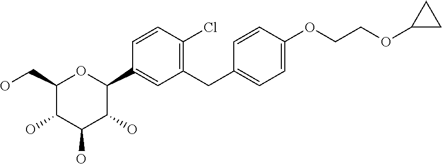 [SMILES: OC[C@H]1O[C@@H](c2ccc(Cl)c(Cc3ccc(OCCOC4CC4)cc3)c2)[C@H](O)[C@@H](O)[C@@H]1O]

(20) Janagliflozin, represented by formula (20):

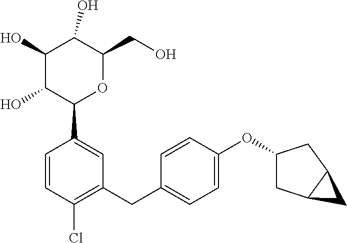 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

(21) Rongliflozin, represented by formula (21):

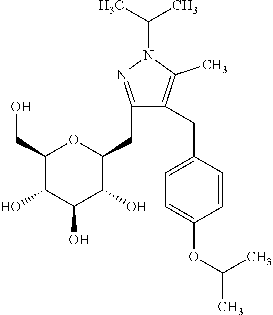 [SMILES: Cc1c(Cc2ccc(OC(C)C)cc2)c(C[C@@H]2O[C@H](CO)[C@@H](O)[C@H](O)[C@H]2O)nn1C(C)C]

(22) Wanpagliflozin.

The term “velagliflozin” as employed herein refers to velagliflozin of the above structure as well as pharmaceutically acceptable forms thereof, including hydrates and solvates thereof, and crystalline forms thereof. The compound, methods of its synthesis and co-crystals thereof are described in WO 2007/128749, WO 2014/016381 and WO 2019/121509 for example.

The term “dapagliflozin” as employed herein refers to dapagliflozin of the above structure as well as pharmaceutically acceptable forms thereof, including hydrates and solvates thereof, and crystalline forms thereof. The compound and methods of its synthesis are described in WO 03/099836 for example. Preferred hydrates, solvates and crystalline forms are described in the patent applications WO 2008/116179 and WO 2008/002824 for example.

The term “canagliflozin” as employed herein refers to canagliflozin of the above structure as well as pharmaceutically acceptable forms thereof, including hydrates and solvates thereof, and crystalline forms thereof. The compound and methods of its synthesis are described in WO 2005/012326 and WO 2009/035969 for example. Preferred hydrates, solvates and crystalline forms are described in the patent application WO 2008/069327 for example.

The term “empagliflozin” as employed herein refers to empagliflozin of the above structure as well as pharmaceutically acceptable forms thereof, including hydrates and solvates thereof, and crystalline forms thereof. The compound and methods of its synthesis are described in WO 2005/092877, WO 2006/120208 and WO 2011/039108 for example. A preferred crystalline form is described in the patent applications WO 2006/117359 and WO 2011/039107 for example.

The term “atigliflozin” as employed herein refers to atigliflozin of the above structure as well as pharmaceutically acceptable forms thereof, including hydrates and solvates thereof, and crystalline forms thereof. The compound and methods of its synthesis are described in WO 2004/007517 for example.

The term “ipragliflozin” as employed herein refers to ipragliflozin of the above structure as well as pharmaceutically acceptable forms thereof, including hydrates and solvates thereof, and crystalline forms thereof. The compound and methods of its synthesis are described in WO 2004/080990, WO 2005/012326 and WO 2007/114475 for example.

The term “tofogliflozin” as employed herein refers to tofogliflozin of the above structure as well as pharmaceutically acceptable forms thereof, including hydrates and solvates thereof, and crystalline forms thereof. The compound and methods of its synthesis are described in WO 2007/140191 and WO 2008/013280 for example.

The term “luseogliflozin” as employed herein refers to luseogliflozin of the above structure as well as pharmaceutically acceptable forms thereof, including hydrates and solvates thereof, and crystalline forms thereof.

The term “ertugliflozin” as employed herein refers to ertugliflozin of the above structure as well as pharmaceutically acceptable forms thereof, including hydrates and solvates thereof, and crystalline forms thereof. The compound is described for example in WO 2010/023594.

The term “remogliflozin” as employed herein refers to remogliflozin of the above structure as well as pharmaceutically acceptable forms thereof, including prodrugs of remogliflozin, in particular remogliflozin etabonate, including hydrates and solvates thereof, and crystalline forms thereof. Methods of its synthesis are described in the patent applications EP 1 213 296 and EP 1 354 888 for example.

The term “sergliflozin” as employed herein refers to sergliflozin of the above structure as well as pharmaceutically acceptable forms thereof, including prodrugs of sergliflozin, in particular sergliflozin etabonate, including hydrates and solvates thereof, and crystalline forms thereof. Methods for its manufacture are described in the patent applications EP 1 344 780 and EP 1 489 089 for example.

The compound of formula (16) above, i.e. sotagliflozin, and its manufacture are described for example in WO 2008/042688 or WO 2009/014970.

Preferred SGLT-2 inhibitors are glucopyranosyl-substituted benzene derivatives. Optionally, one or more hydroxyl groups of the glucopyranosyl group in such one or more SGLT-2 inhibitors may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

More preferred are glucopyranosyl-substituted benzonitrile derivatives of formula (1) as disclosed herein above. Yet more preferred are glucopyranosyl-substituted benzonitrile derivatives of formula (18):

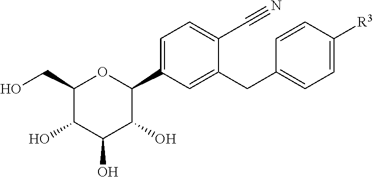

wherein
R denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetra-hydrofuran-3-yloxy or (S)-tetrahydrofuran-3-yloxy or cyano; and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
   or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Preferably, such SGLT-2 inhibitor is velagliflozin as shown in formula (2). Optionally, one or more hydroxyl groups of the β-D-glucopyranosyl group of velagliflozin may be acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl.

Thus, in a preferred embodiment, the at least one SGLT-2 inhibitor according to the present invention is a glucopyranosyl-substituted benzene derivative SGLT-2 inhibitor, preferably a SGLT-2 inhibitor of formula (1), more preferably of formula (18), or yet more preferably of formula (2), i.e. velagliflozin, in each case as defined herein above.

Herein, references to SGLT-2 inhibitors and/or their use according to the invention encompass pharmaceutically acceptable forms of the SGLT-2 inhibitors, unless otherwise stated.

According to the invention, any pharmaceutically acceptable form of the SGLT-2 inhibitor, e.g. of formula (1), preferably formula (18), more preferably formula (2), may be used. E.g. a crystalline form may be used. Prodrug forms are also encompassed by the present invention.

Prodrug forms may include, e.g., esters and/or hydrates. The term “prodrug” is also meant to include any covalently bonded carrier, which releases the active compound of the invention in vivo when the prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention.

Crystalline forms for use according to the invention include a complex of an SGLT-2 inhibitor with one or more amino acids (see e.g. WO 2014/016381)-so-called co-crystals. An amino acid for such use may be a natural amino acid. The amino acid may be a proteogenic amino acid (including L-hydroxyproline), or a non-proteogenic amino acid. The amino acid may be a D- or an L-amino acid. In some preferred embodiments, the amino acid is proline (L-proline and/or D-proline, preferably L-proline). E.g., a crystalline complex/co-crystal of velagliflozin with proline (e.g. L-proline) and crystalline water is preferred.

Thus, herein is disclosed a crystalline complex/co-crystal between one or more natural amino acids and an SGLT-2 inhibitor, e.g., a crystalline complex/co-crystal between one or more natural amino acids and a glu-copyranosyl-substituted benzene derivative SGLT-2 inhibitor, preferably a SGLT-2 inhibitor of formula (1), more preferably of formula (18) or yet more preferably of formula (2) (velagliflozin).

A certain pharmaceutical activity is the basic prerequisite to be fulfilled by a pharmaceutically active agent before it is approved as a medicament on the market. However, there is a variety of additional requirements a pharmaceutically active agent has to comply with. These requirements are based on various parameters, which are connected with the nature of the active substance itself. Without being restrictive, examples of these parameters are the stability of the active agent under various environmental conditions, its stability during production of the pharmaceutical formulation and the stability of the active agent in the final medicament compositions. The pharmaceutically active substance used for preparing the pharmaceutical compositions should be as pure as possible and its stability in long-term storage must be guaranteed under various environmental conditions. This is essential to prevent the use of pharmaceutical compositions, which contain, in addition to the actual active substance, breakdown products thereof, for example. In such cases, the content of active substance in the medicament might be less than that specified.

Uniform distribution of the medicament in the formulation is a critical factor, particularly when the medicament has to be given in low doses. To ensure uniform distribution, the particle size of the active substance can be reduced to a suitable level, e.g. by grinding. Since breakdown of the pharmaceutically active substance as a side effect of the grinding (or micronizing) has to be avoided as far as possible, in spite of the hard conditions required during the process, it is essential that the active substance should be highly stable throughout the grinding process. Only if the active substance is sufficiently stable during the grinding process it is possible to produce a homogeneous pharmaceutical formulation, which always contains the specified amount of active substance in a reproducible manner.

Another problem, which may arise in the grinding process for preparing the desired pharmaceutical formulation, is the input of energy caused by this process and the stress on the surface of the crystals. This may in certain circumstances lead to polymorphous changes, to amorphization or to a change in the crystal lattice. Since the pharmaceutical quality of a pharmaceutical formulation requires that the active substance should always have the same crystalline morphology, the stability and properties of the crystalline active substance are subject to stringent requirements from this point of view as well.

The stability of a pharmaceutically active substance is also important in pharmaceutical compositions for determining the shelf life of the particular medicament; the shelf life is the length of time during which the medicament can be administered without any risk. High stability of a medicament in the abovementioned pharmaceutical compositions under various storage conditions is therefore an additional advantage for both the patient and the manufacturer.

The absorption of moisture reduces the content of pharmaceutically active substance because of the increased weight caused by the uptake of water. Pharmaceutical compositions with a tendency to absorb moisture have to be protected from moisture during storage, e.g. by the addition of suitable drying agents or by storing the drug in an environment where it is protected from moisture. Preferably, therefore, a pharmaceutically active substance should be at best slightly hygroscopic.

Furthermore, the availability of a well-defined crystalline form allows the purification of the drug substance by recrystallization.

Apart from the requirements indicated above, it should be generally borne in mind that any change to the solid state of a pharmaceutical composition, which is capable of improving its physical and chemical stability, gives a significant advantage over less stable forms of the same medicament.

A crystalline complex/co-crystal between a natural amino acid and an SGLT-2 inhibitor (e.g. a glucopyranosyl-substituted benzene derivative or a SGLT-2 inhibitor of formula (1), or formula (18) or, particularly, of formula (2), i.e. velagliflozin) fulfills important requirements mentioned hereinbefore.

SGLT-2 inhibitors for use according to the invention may be prepared as pharmaceutical compositions. They may be prepared as solid or as liquid formulations. In either case, they are preferably prepared for oral administration, preferably in liquid form for oral administration (see e.g. WO 2017/032799). The SGLT-2 inhibitors may, however, also be prepared, e.g., for parenteral administration. Solid formulations include tablets, granular forms, and other solid forms such as suppositories. Among solid formulations, tablets and granular forms are preferred.

Pharmaceutical compositions within the meaning of the present invention may comprise an SGLT-2 inhibitor according to the present invention and one or more excipients. Any excipient that allows for, or supports, the intended medical effect may be used. Such excipients are available to the skilled person. Useful excipients are for example anti-adherents (used to reduce the adhesion between the powder (granules) and the punch faces and thus prevent sticking to tablet punches), binders (solution binders or dry binders that hold the ingredients together), coatings (to protect tablet ingredients from deterioration by moisture in the air and make large or unpleasant-tasting tablets easier to swallow), disintegrants (to allow the tablet to break upon dilution), fillers, diluents, flavours, colours, glidants (flow regulators-to promote powder flow by reducing interparticle friction and cohesion), lubricants (to prevent ingredients from clumping together and from sticking to the tablet punches or capsule filling machine), preservatives, sorbents, sweeteners etc.

Formulations according to the invention, e.g. solid formulations, may comprise carriers and/or disintegrants selected from the group of sugars and sugar alcohols, e.g. mannitol, lactose, starch, cellulose, microcrystalline cellulose and cellulose derivatives, e.g. methylcellulose, and the like.

Manufacturing procedures for formulations suitable for canines are known to the person skilled in the art, and for solid formulations comprise, e.g., direct compression, dry granulation and wet granulation. In the direct compression process, the active ingredient and all other excipients are placed together in a compression apparatus that is directly applied to press tablets out of this material. The resulting tablets can optionally be coated afterwards in order to protect them physically and/or chemically, e.g. by a material known from the state of the art.

A unit for administration, e.g. a single liquid dose or a unit of a solid formulation, e.g. a tablet, may comprise 0.1 mg to 10 mg, or e.g. 0.3 mg to 1 mg, 1 mg to 3 mg, 3 mg to 10 mg; or 5 to 2500 mg, or e.g. 5 to 2000 mg, 5 mg to 1500 mg, 10 mg to 1500 mg, 10 mg to 1000 mg, or 10-500 mg of an SGLT-2 inhibitor for use according to the invention. As the skilled person would understand, the content of the SGLT-2 inhibitor in a solid formulation, or any formulation as disclosed herein for administration to a non-human mammal, such as a carnivore, in particular a cat and/or a dog, may be increased or decreased as appropriate in proportion to the body weight of such non-human mammal to be treated.

In one embodiment, a pharmaceutical composition for use according to the invention is designed for oral or parenteral administration, preferably for oral administration. Especially the oral administration is ameliorated by excipients, which modify the smell and/or haptic properties of the pharmaceutical composition for the intended patient, e.g. as described.

When the SGLT-2 inhibitor for use according to the invention is formulated for oral administration, it is preferred that excipients confer properties, e.g. palatability and/or chewability that render the formulation suitable for administration to a canine animal.

Also preferred are liquid formulations. Liquid formulations may be, e.g., solutions, syrups or suspensions. They may be administered directly to the non-human mammal, e.g. canines, or may be mixed with the food and/or drink (e.g. drinking water, or the like) of the non-human mammal, e.g. canine animal. One advantage of a liquid formulation (similar to a formulation in granular form), is that such a dosage form allows precise dosing. For example, the SGLT-2 inhibitor may be dosed precisely in proportion to the body mass of a non-human mammal, e.g. canine animal. Typical compositions of liquid formulations are known to the person skilled in the art.

A practitioner skilled in the art can determine suitable doses for the uses of the present invention. Preferred units dosing units include mg/kg bodyweight, i.e. mg SGLT-2 inhibitor per body mass of the non-human mammal. An SGLT-2 inhibitor of the invention may, e.g., be administered in doses of 0.01-10 mg/kg bodyweight per day, e.g. 0.01-5 mg/kg bodyweight per day, e.g. 0.01-4 mg/kg bodyweight per day, e.g. 0.01-3 mg/kg bodyweight per day, e.g. 0.01-2 mg/kg bodyweight per day, e.g. 0.01-1.5 mg/kg bodyweight per day, e.g., 0.01-1 mg/kg bodyweight per day, e.g. 0.01-0.75 mg/kg bodyweight per day, e.g. 0.01-0.5 mg/kg body-weight per day, e.g. 0.01-0.4 mg/kg bodyweight per day; or 0.1 to 3.0 mg/kg bodyweight per day, preferably from 0.2 to 2.0 mg/kg bodyweight per day, more preferably from 0.1 to 1 mg/kg bodyweight per day or from 0.5 to 1 mg/kg bodyweight per day. In another preferred embodiment, the dose is 0.01-1 mg/kg bodyweight per day, preferably 0.01-0.5 mg/kg bodyweight per day, more preferably 0.02-0.4 mg/kg bodyweight per day, e.g. 0.03-0.3 mg/kg bodyweight per day.

A practitioner skilled in the art is able to prepare an SGLT-2 inhibitor of the invention for administration according to a desired dose.

### EXAMPLES

The following examples serve to further illustrate the present invention; but the same should not be construed as a limitation of the scope of the invention disclosed herein.

### Example 1 Exploratory Clinical Field Study—Cats

Client owned patients diagnosed with CKD (IRIS stage 3) diagnosed via physical examination and biomarker levels are orally treated once daily with velagliflozin 0.1 mg/kg bodyweight. During the study period the body weight, body condition score, biomarker levels (creatinine and SDMA), presence of concomitant conditions and need for further medication are examined on regular basis during the visits at the site by the investigator. The parameters examined are creatinine and SDMA levels, proteinuria, blood pressure, quality of life, and hydration status. Additionally, variables as the delayed onset of renal failure, survival time of the cat patients, IRiS stage progression and renal related mortality and/or morbidity are observed. Study results confirm a beneficial effect on renal function and a delay in progression of the disease.

### Example 2 Exploratory Clinical Field Study—Dogs

Client owned patients diagnosed with CKD (IRIS stage 3) diagnosed via physical examination and biomarker levels are orally treated once daily with velagliflozin 0.3 mg/kg bodyweight. During the study period the body weight, body condition score, biomarker levels (creatinine and SDMA), proteinuria, presence of concomitant conditions and need for further medication are examined on regular basis during the visits at the site by the investigator. The parameters examined are creatinine and SDMA levels, proteinuria, blood pressure and hydration status. Additionally, variables as the delayed onset of renal failure, survival time of the dog patients, IRiS stage progression and renal related mortality and/or morbidity are observed. Study results confirm a beneficial effect on renal function and a delay in progression of the disease.

### Example 3 Exploratory Clinical Field Trial in Cats With CKD

The clinical trial was conducted at three animal clinics. Overall, 9 cats with chronic kidney disease (CKD) were included in the study and treated once daily orally with 1 mg/kg bodyweight velagliflozin for up to 150 days (depending on the time of enrollment).

Diagnosis of CKD was based on two measurements of serum creatinine concentration at least 2 weeks apart from each other (historical value and screening, and value at screening in fasted cats), as recommended by the International Renal Interest Society (IRIS 2019). In order to qualify for enrollment, serum creatinine levels needed to be ≥2.2 mg/dL, but <5.0 mg/dL.

Disease staging throughout the study was performed based on serum creatinine levels in accordance with the IRIS CKD staging (IRIS 2019).

At enrollment, 5 of 9 cats were in stage 2 (cases 200-001, 200-005, 200-006, 300-003, 300-008) and 4 of 9 cats were in stage 3 (cases 200-003, 200-004, 200-007, 400-004).

### Case 400-004—Improved:

Case 400-004 is a 12-year-old, castrated, 6.9 kg, European cat on a renal diet. According to the owner, appetite was normal, drinking behavior was excessive and overall quality of life was good at the time of screening. The cat did not receive any medication at screening. One non-serious adverse event was reported (owner reported diarrhea). Recovery occurred within 24 hours without treatment. The cat received no concomitant treatment throughout the study.

As a result of the oral treatment with 1 mg/kg bodyweight velagliflozin once daily clinical signs as well as biochemical parameters demonstrated an improvement of the condition (table 1 and 2). This was based on a decrease in serum creatinine and BUN concentration. Serum potassium and phosphate levels remained within the reference range. In addition, total urine protein reduced, while UPC ratio remained in the normal range. Drinking behavior improved from excessive to normal according to the owner. The expected glucosuric effect of velagliflozin under treatment was confirmed.

Case 400-004 completed the study.

TABLE 1
Laboratory values and IRIS stage at study visits - case 400-004

| | | | IRIS | | | | |
|---|---|---|---|---|---|---|---|
| | | Serum | Stage | | | | |
| | BUN | creatinine | (based on | Potassium | | Phosphorus | Hematocrit |
| | (mg/dL) | (mg/dL) | serum | (mmol/L) | | (mmol/L) | (%) |
| | [16-38 | [0.9-2.3 | creatinine | [3.3-5.8 | UPC | [0.8-2.2 | [28.2- |
| | mg/dL] | mg/dL] | level) | mmol/L] | [<0.33] | mmol/L] | 52.7%] |
| Screening | 20.2 | 4.2 | 3 | 4.3 | 0.1 | 1.4 | 44.0% |
| visit | | | | | | | |
| Visit 1 | 20.0 | 4.0 | 3 | 3.9 | — | 1.3 | 41.0% |
| (Day 30) | | | | | | | |
| Visit 2 | 19.3 | 3.2 | 3 | 3.8 | 0.1 | 1.3 | 45.0% |
| (Day 90) | | | | | | | |
| Visit 3 | 24.3 | 2.9 | 3 | 4.0 | — | 1.5 | 45.0% |
| (Day 119) | | | | | | | |

TABLE 2
Clinical parameters at study visits - case 400-004

| | Systolic | Owner | Owner | Owner |
|---|---|---|---|---|
| | blood | assessment | assessment of | assessment - |
| | pressure | of the | the drinking | quality of |
| | (mmHg) | appetite | behavior | life |
| Screening visit | 151.3 | normal | excessive | good |
| Visit 1 | 142.7 | normal | normal | good |
| (Day 30) | | | | |
| Visit 2 | 157.3 | poor | normal | good |
| (Day 90) | | | | |
| Visit 3 | 149.3 | normal | normal | good |
| (Day 119) | | | | |

### Case 300-008—Stable:

Case 300-008 is a 12-year-old, 2.5 kg, castrated European cat on a standard diet. According to the owner, appetite was normal, drinking behavior was excessive and overall quality of life was good at the time of screening. At screening, the cat did not receive any medication. No adverse event was reported during the study. The cat received no concomitant treatment throughout the study.

As a result of the treatment with 1 mg/kg bodyweight velagliflozin per os once daily clinical signs as well as biochemical parameters demonstrated a stabilization of the condition. Serum creatinine concentration remained stable, and serum potassium and phosphate levels remained in the reference range (table 3). In accordance, total urine protein and UPC ratio remained within the reference range. Drinking behavior improved from excessive to normal according to owner's assessment (table 4). The expected glucosuric effect of velagliflozin under treatment was confirmed.

Case 300-008 completed the study.

TABLE 3
Laboratory values and IRIS stage at study visits - case 300-008

| | | Serum | IRIS | | | | |
|---|---|---|---|---|---|---|---|
| | BUN | creatinine | Stage | Potassium | | Phosphorus | Hematocrit |
| | (mg/dL) | (mg/dL) | (based on | (mmol/L) | | (mmol/L) | (%) |
| | [16-38 | [0.9-2.3 | creatinine | [3.3-5.8 | UPC | [0.8-2.2 | [28.2- |
| | mg/dL] | mg/dL]) | level) | mmol/L] | [<0.33] | mmol/L] | 52.7%] |
| Screening | 36 | 2.4 | 2 | 4.9 | 0.2 | 1.3 | 26.7 |
| visit | | | | | | | |
| Visit 1 | 65 | 1.9 | 2 | 4.9 | — | 1.4 | 28.8 |
| (Day 30) | | | | | | | |
| Visit 2 | 64 | 2.4 | 2 | 4.4 | 0.2 | 1.5 | 32.4 |
| (Day 90) | | | | | | | |
| Visit 3 | 54 | 2.6 | 2 | 4.4 | 0.3 | 1.6 | 28.2 |
| (Day 103) | | | | | | | |

TABLE 4
Clinical parameters at study visits - case 300-008

| | Systolic | Owner | Owner | Owner |
|---|---|---|---|---|
| | blood | assessment | assessment of | assessment - |
| | pressure | of the | the drinking | quality of |
| | (mmHg) | appetite | behavior | life |
| Screening | 148.0 | normal | excessive | good |
| visit | | | | |
| Visit 1 | 150.0 | poor | normal | good |
| (Day 30) | | | | |
| Visit 2 | 150.0 | normal | unknown | good |
| (Day 90) | | | | |
| Visit 3 | 148.7 | normal | normal | good |
| (Day 103) | | | | |

### Case 200-004—Improved:

Case 200-004 is a 14-year-old, 3.9 kg, castrated Persian cat on a renal diet, diagnosed with chronic kidney disease, IRIS stage 3. According to the owner, appetite was normal, drinking behavior was normal and overall quality of life was very good at the time of screening. The cat was reported with three pre-existing medical conditions, witnessed in the 3 months prior to screening: tartar, haematuria and erythrocytopenia, all ongoing at screening. The cat consequently received butylscopolamine and beta-glucan-methonin for 4 days (from Day 0 to Day 3) and 20 days (from Day 1 to 20), respectively. No adverse event was reported during the study.

As a result of the treatment with 1 mg/kg bodyweight velagliflozin per os once daily clinical signs remained normal while biochemical parameters indicated improvement of the condition. Serum creatinine decreased by a clinically relevant amount, consequently improving the IRIS stage of the disease (from 3 to 2 (Table 5)).

Quality of life remained very good according to the owner. The cat's appetite and drinking behavior remained normal throughout the study. (Table 6).

Case 200-004 completed the study.

TABLE 5
Laboratory values and IRIS stage at study visits - case 200-004

| | | | IRIS | | | | |
|---|---|---|---|---|---|---|---|
| | | Serum- | Stage | | | | |
| | BUN | creatinine | (based on | Potassium | | Phosphorus | Hematocrit |
| | (mg/dL) | (mg/dL) | serum | (mmol/L) | | (mmol/L) | (%) |
| | [16-38 | [0.9-2.3 | creatinine | [3.3-5.8 | UPC | [0.8-2.2 | [28.2- |
| | mg/dL] | mg/dL] | level) | mmol/L] | [<0.33] | mmol/L] | 52.7%] |
| Screening | 46 | 2.9 | 3 | 4.9 | 0.1 | 1.2 | 33.4 |
| visit | | | | | | | |
| Visit 1 | 42 | 2.7 | 2 | 4.5 | — | 1.3 | 37.0 |
| (Day 30) | | | | | | | |
| Visit 2 | 36 | 2.5 | 2 | 4.9 | — | 1.3 | 32.9 |
| (Day 90) | | | | | | | |
| Visit 3 | 38 | 2.6 | 2 | 4.9 | — | 1.2 | 34.8 |
| (Day 143) | | | | | | | |

TABLE 6
Clinical parameters at study visits - case 200-004

| | Systolic | Owner | Owner | Owner |
|---|---|---|---|---|
| | blood | assessment | assessment of | assessment - |
| | pressure | of the | the drinking | quality of |
| | (mmHg) | appetite | behavior | life |
| Screening visit | 130.7 | normal | normal | very good |
| Visit 1 | 148.0 | normal | normal | very good |
| (Day 30) | | | | |
| Visit 2 | 129.3 | normal | normal | very good |
| (Day 90) | | | | |
| Visit 3 | 148.7 | normal | normal | very good |
| (Day 143) | | | | |

### Case 200-005—Stable:

Case 200-005 is an 8-year-old, 7.0 kg, castrated Savannah cat on a standard diet, diagnosed with chronic kidney disease, IRIS stage 2. According to the owner, appetite and drinking behavior was normal, and overall quality of life was very good at the time of screening. One pre-existing medical condition (in the previous 3 months) was reported at screening: loose tooth. The cat received no concomitant treatment throughout the study. No adverse event was reported following inclusion.

As a result of the treatment with 1 mg/kg bodyweight velagliflozin per os once daily clinical signs as well as biochemical parameters demonstrated a stabilization of the condition. Serum creatinine decreased, while BUN remained stable (Table 7). Serum potassium levels remained stable. Quality of life was classified to be good according to the owner. The cat's appetite and drinking behavior remained normal throughout the study (Table 8). The expected glucosuric effect of velagliflozin under treatment was confirmed. In accordance, total urine protein reduced, while UPC ratio remained at 0.1.

Case 200-005 completed the study.

TABLE 7
Laboratory values and IRIS stage at study visits - case 200-005

| | | Serum | IRIS | | | | |
|---|---|---|---|---|---|---|---|
| | BUN | creatinine | Stage | Potassium | | Phosphorus | Hematocrit |
| | (mg/dL) | (mg/dL) | (based on the | (mmol/L) | | (mmol/L) | (%) |
| | [16-38 | [0.9-2.3 | creatinine | [3.3-5.8 | UPC | [0.8-2.2 | [28.2- |
| | mg/dL] | mg/dL] | level) | mmol/L] | [<0.33] | mmol/L] | 52.7%] |
| Screening | 37 | 2.2 | 2 | 4.9 | 0.1 | — | 42.3 |
| visit | | | | | | | |
| Visit 1 | 37 | 2.0 | 2 | 4.0 | — | 1.0 | 45.1 |
| (Day 30) | | | | | | | |
| Visit 2 | 32 | 1.9 | 2 | 3.9 | 0.1 | 1.3 | 39.2 |
| (Day 90) | | | | | | | |
| Visit 3 | 37 | 1.9 | 2 | 4.0 | — | 1.1 | 40.4 |
| (Day 128) | | | | | | | |

TABLE 8
Clinical parameters at study visits - case 200-005

| | Systolic | Owner | Owner | Owner |
|---|---|---|---|---|
| | blood | assessment | assessment of | assessment - |
| | pressure | of the | the drinking | quality of |
| | (mmHg) | appetite | behavior | life |
| Screening visit | 139.3 | normal | normal | very good |
| Visit 1 | 148.0 | normal | normal | very good |
| (Day 30) | | | | |
| Visit 2 | 130.0 | normal | normal | good |
| (Day 90) | | | | |
| Visit 3 | 123.3 | normal | normal | good |
| (Day 128) | | | | |

### Case 200-001—Stable:

Case 200-001 is a 14-year-old, 2.8 kg, castrated Persian cat on renal diet, diagnosed with chronic kidney disease, IRIS stage 2. According to the owner, appetite and drinking behavior were normal, and overall quality of life was very good at the time of screening. Three pre-existing medical condition (in the previous 3 months) were reported at screening: cataract, dental tartar, vomitus. Two adverse events were reported for this patient: pectin responsive diarrhea for one day and pancreatitis, which was treated with amoxicillin, prednisolone and phosphate blocker. The patient was removed from the study early for pancreatitis, which is a known concomitant disease in elderly cats.

As a result of the treatment with 1 mg/kg bodyweight velagliflozin per os once daily clinical signs as well as biochemical parameters demonstrate a stabilization of the condition. Serum creatinine remained stable with one single measurement on Day 90 being increased, while BUN remained stable (Table 9). Serum potassium levels decreased within the reference range. The expected glucosuric effect of velagliflozin was confirmed. In accordance, total urine protein reduced. UPC improved from 0.4 to 0.2. Quality of life remained very good according to the owner. The cat's appetite and drinking behavior remained normal throughout the study (Table 10).

TABLE 9
Laboratory values and IRIS stage at study visits - case 200-001

| | | Serum | IRIS | | | | |
|---|---|---|---|---|---|---|---|
| | BUN | creatinine | Stage | Potassium | | Phosphorus | Hematocrit |
| | (mg/dL) | (mg/dL) | (based on the | (mmol/L) | | (mmol/L) | (%) |
| | [16-38 | [0.9-2.3 | creatinine | [3.3-5.8 | UPC | [0.8-2.2 | [28.2- |
| | mg/dL] | mg/dL]) | level) | mmol/L] | [<0.33] | mmol/L] | 52.7%] |
| Screening | 39 | 2.6 | 2 | 5.2 | 0.4 | 1.4 | 37.4 |
| visit | | | | | | | |
| Visit 1 | 38 | 2.6 | 2 | 4.6 | — | 1.3 | 38.5 |
| (Day 30) | | | | | | | |
| Visit 2 | 47 | 3.2 | 3 | 4.3 | 0.2 | 2.4 | 34.6 |
| (Day 90) | | | | | | | |
| Visit 3 | 41 | 2.6 | 2 | 4.3 | — | 1.6 | 32.6 |
| (Day 111) | | | | | | | |

TABLE 10
Clinical parameters at study visits - case 200-001

| | Systolic | Owner | Owner | Owner |
|---|---|---|---|---|
| | blood | assessment | assessment of | assessment - |
| | pressure | of the | the drinking | quality of |
| | (mmHg) | appetite | behavior | life |
| Screening visit | 152.0 | normal | normal | very good |
| Visit 1 | 126.7 | normal | normal | very good |
| (Day 30) | | | | |
| Visit 2 | 122.7 | normal | normal | very good |
| (Day 90) | | | | |
| Visit 3 | 131.3 | normal | normal | very good |
| (Day 111) | | | | |

### Case 200-006—Stable:

Case 200-006 is a 16-year-old, 3.7 kg, castrated European cat on standard diet, diagnosed with chronic kidney disease. IRIS stage 2. According to the owner, appetite was poor, while drinking behavior was excessive, and overall quality of life was good at the time of screening. In regard to pre-existing medical condition (in the previous 3 months) the following was reported at screening: weight loss, delayed eating, cataract, gingivitis. Three adverse events were reported: diarrhoea, vomitus as well as cystitis. Diarrhoea was treated with pectin and was resolved within 24 hours. Vomiting was treated with barium sulfate, as well as a single injection of glucose, saline, vitamin complex and maropitant. Cystitis was diagnosed shortly prior to visit 2 and was treated with amoxicillin and metamizole. Diarrhoea resolved after 3 days, cystitis after 2 days, respectively.

As a result of the treatment with 1 mg/kg bodyweight velagliflozin per os once daily clinical signs as well as biochemical parameters demonstrate a stable condition. Serum creatinine and BUN remained stable (table 11). Serum potassium levels remained within the reference range. The expected glucosuric effect of velagliflozin under treatment was confirmed. Total urine protein and UPC remained within the reference range. Quality of life remained good according to the owner. The cat's appetite improved to normal and drinking behavior improved to normal but worsened again to excessive (potentially due to cystitis) (table 12).

Overall, the cat's condition remains stable. The worsening of the parameters proteinuria and water uptake under the treatment with velagliflozin at visit 2 and 3 may be related to cystitis.

Case 200-006 completed the study.

TABLE 11
Laboratory values and IRIS stage at study visits - case 200-006

| | | Serum | IRIS | | | | |
|---|---|---|---|---|---|---|---|
| | BUN | creatinine | Stage | Potassium | | Phosphorus | Hematocrit |
| | (mg/dL) | (mg/dL) | (based on the | (mmol/L) | | (mmol/L) | (%) |
| | [16-38 | [0.9-2.3 | creatinine | [3.3-5.8 | UPC | [0.8-2.2 | [28.2- |
| | mg/dL] | mg/dL] | level) | mmol/L] | [<0.33] | mmol/L] | 52.7%] |
| Screening | 34 | 2.6 | 2 | 3.8 | 0.1 | 1.3 | 37.9 |
| visit | | | | | | | |
| Visit 1 | 41 | 2.7 | 2 | 3.9 | — | 1.9 | 38.1 |
| (Day 30) | | | | | | | |
| Visit 2 | 30 | 2.7 | 2 | 4.4 | 0.2 | 1.3 | 37.3 |
| (Day 90) | | | | | | | |
| Visit 3 | 42 | 2.8 | 2 | 4.3 | — | 2.0 | 35.5 |
| (Day 121) | | | | | | | |

TABLE 12
Clinical parameters at study visits - case 200-006

| | Systolic | Owner | Owner | Owner |
|---|---|---|---|---|
| | blood | assessment | assessment of | assessment - |
| | pressure | of the | the drinking | quality of |
| | (mmHg) | appetite | behavior | life |
| Screening visit | 141.3 | poor | excessive | good |
| Visit 1 | 139.3 | normal | normal | good |
| (Day 30) | | | | |
| Visit 2 | 155.3 | normal | excessive | good |
| (Day 90) | | | | |
| Visit 3 | 140.0 | normal | excessive | good |
| (Day 121) | | | | |

### Case 200-007—Stable:

Case 200-007 is an 18-year-old, 4.1 kg, castrated European cat on standard diet, diagnosed with chronic kidney disease, IRIS stage 3. According to the owner, appetite was normal, while drinking behavior was excessive, and overall quality of life was very good at the time of screening. The following pre-existing medical conditions (in the previous 3 months) were reported at screening: weight loss, pigmented increase in circumference at the tip of the left ear, cataract, lymphocytopenia and leukocytopenia. Adverse events reported for this patient included three occasions of vomitus, two occasions of inappetence and one occasion of mild anaemia, leukocytopenia. Treatment of vomiting and inappetence included single administration of saline, glucose, maropitant, prednisolone, butylscopolamin, cyproheptadine hydrochlorid, omeprazole and amoxicillin clavulanic acid.

As a result of the treatment with 1 mg/kg bodyweight velagliflozin per os once daily drinking behavior improved to normal, while quality of life changed from very good to good (Table 14). Serum creatinine levels increased mildly (Table 13). Serum potassium levels remained stable within the reference range. The expected glucosuric effect of velagliflozin under treatment was confirmed. Total urine protein and UPC mildly increased.

Based on the more advanced stage of CKD, the cat's condition can be considered stable and within the expected range for the evaluated disease.

Case 200-007 completed the study.

TABLE 13
Laboratory values and IRIS stage at study visits - case 200-007

| | | Serum | IRIS | | | | |
|---|---|---|---|---|---|---|---|
| | BUN | creatinine | Stage | Potassium | | Phosphorus | Hematocrit |
| | (mg/dL) | (mg/dL) | (based on the | (mmol/L) | | (mmol/L) | (%) |
| | [16-38 | [0.9-2.3 | creatinine | [3.3-5.8 | UPC | [0.8-2.2 | [28.2- |
| | mg/dL] | mg/dL] | level) | mmol/L] | [<0.33] | mmol/L] | 52.7%] |
| Screening | 41 | 2.9 | 3 | 4.4 | 0.1 | 1.2 | 37.6 |
| visit | | | | | | | |
| Visit 1 | 40 | 3.2 | 3 | 4.0 | — | 1.0 | 35.9 |
| (Day 30) | | | | | | | |
| Visit 2 | 42 | 3.3 | 3 | 4.4 | 0.2 | 1.3 | 32.6 |
| (Day 90) | | | | | | | |
| Visit 3 | 43 | 3.2 | 3 | 3.9 | — | 1.3 | 28.9 |
| (Day 114) | | | | | | | |

TABLE 14
Clinical parameters at study visits - case 200-007

| | Systolic | Owner | Owner | Owner |
|---|---|---|---|---|
| | blood | assessment | assessment of | assessment - |
| | pressure | of the | the drinking | quality of |
| | (mmHg) | appetite | behavior | life |
| Screening visit | 139.3 | normal | excessive | very good |
| Visit 1 | 122.7 | normal | normal | very good |
| (Day 30) | | | | |
| Visit 2 | 130.7 | normal | normal | good |
| (Day 90) | | | | |
| Visit 3 | 130.7 | normal | normal | good |
| (Day 114) | | | | |

### Summary:

Chronic kidney disease is a progressive disease, characterized by declining renal function. Affected cats show an unpredictable time course of the disease with periods of stable renal function before decompensation. Disease-related azotemia and clinical signs become apparent only late, when 75% or more of the kidney tissue is impacted. Improvement in renal function cannot be expected. Improvement in clinical signs is not expected due to the progression of the disease, unless renal treatment occurs.

CKD-associated complications include systemic hypertension, secondary renal hyperparathyroidism, hypokalaemia, anaemia, and metabolic acidosis. Identified risk factors for decreased survival time of CKD patients include increased UPC, plasma creatinine, phosphate (measured as correlating phosphorus) or urea, high blood leucocyte counts, as well as signs of anemia (low haemoglobin and haematocrit). To date, treatment goals in CKD patients are:
slow disease progression (correct underlaying cause, stabilize identified risk factors, treat associated complications)
ensure quality of life (minimize clinical signs)

The study enrolled 9 cats with mild to moderate azotemia (advanced IRIS stage 2 and IRIS stage 3). For all cats except cat 200-004 (no data recorded on total protein and UPC following treatment start), the expected glucosuric effect of velagliflozin under treatment was confirmed. Despite from renal diet or phosphate binders (patient had to be stable on diet/phosphate binder prior screening), no concomitant renal therapy was administered to the cats.

Two cats were removed early from the study already on day 30. Since these cats were treated only a short time and had only one of two recommended measurements of the relevant laboratory parameters under treatment of velagliflozin available, they were no further assessed.
Case 200-003 is a 15-year-old, 3.9 kg, castrated European cat on renal diet, diagnosed with chronic kidney disease in a more advanced stage of CKD (IRIS stage 3) at the time of enrollment. Unfortunately, the cat was removed early, based on the worsening of a single measurement of laboratory parameters (which is expected in IRIS stage 3 cats), despite good quality of life and appetite and drinking behavior improving to normal under velagliflozin treatment.
Case 300-003 is a 13-year-old, 4.3 kg, castrated European cat on renal diet, diagnosed with chronic kidney disease. IRIS stage 2. According to the owner, appetite and drinking behavior was normal, and overall quality of life was very good at the time of screening. Tooth pain, anorexia and feline odontoclastic-resorptive lesions (FORL) were reported for this cat and treated with tooth extraction/cleaning under anaesthesia (anaesthesia, antibiotic and pain medication administered), followed by the early removal of the patient despite stable laboratory parameters and no worsening of the evaluated clinical signs.

All other cats were treated for >90 days had at least data for two time points under treatment available and were therefore evaluated. Progression of the disease, as well as IRIS staging in this study was based on the most reliable biomarker: serum creatinine.

Out of the remaining seven cats, 3 cats unexpectedly showed a decrease in serum creatinine of ≥10% within the observation period. Improvement in serum creatinine in these cats was accompanied by improved or stable status of the majority of other kidney relevant laboratory parameters and/or clinical signs. IRIS staging improved by one category in one of these cats, which is extremely unexpected in an advanced stage of the disease (stage 3->stage 2), since disease progression is anticipated. This in particular confirms the positive effect of 1 mg/kg bodyweight velagliflozin treatment per os once daily, because the cat did not receive any renal standard of care treatment.

The other cats (4 cats) showed stable condition of laboratory parameters related to CKD (serum creatinine) and clinical signs.

There were no complications of CKD observed under oral treatment with 1 mg/kg bodyweight velagliflozin once daily.

Clinical signs related to CKD either remained good/stable or improved in the majority of cats. Particularly improvement in clinical signs is not expected in CKD patients, unless renal treatment is administered.

Risk factors of progression in CKD should be prevented, which include serum phosphate levels, serum potassium levels, hematocrit, UPC and blood pressure. In this study the following observations were made:
Serum phosphorus concentration:
   There is strong evidence in the literature indicating that maintaining plasma phosphorus concentrations to within the International Renal Interest Society targets for CKD patients improves survival time. In this study 5 cats received a low phosphate diet versus 4 cats that received a standard diet. In all evaluated cases in this clinical trial, independent of the administration of low phosphate diet and/or phosphate binders, serum phosphate levels remained within the laboratories reference range throughout the study (except for one measurement for case 200-001 on day 90), suggesting a beneficial effect of velagliflozin on phosphorus homeostasis and therefore, a benefit in CKD cats.
Serum potassium levels stayed within the reference range in all treated cats, suggesting the prevention of hypokalemia, a known complication in CKD cats, by oral treatment of 1 mg/kg bodyweight velagliflozin once daily.
Hematocrit was monitored for the detection of anemia of renal disease. Hematocrit remained within the reference range in all cats under oral treatment with 1 mg/kg bodyweight velagliflozin, suggesting an anaemia preventing effect.
Proteinuria remained within the rererence range of ≤0.33 in all patients suggesting prevention of progression of proteinuria by oral treatment of 1 mg/kg bodyweight velagliflozin once daily.
Systolic blood pressure remained <160 mmHg during the course of the study in all cats.

### Conclusion:

Velagliflozin administered orally at a dose of 1 mg/kg bodyweight once daily to feline CKD patients stabilized or even improved renal parameters and clinical signs in IRIS stage 2 and 3 CKD cats (e.g., UPC improved in case 200-001, appetite normalized in case 200-006, drinking behavior normalized in cases 200-007, 400-004, and 300-008). Serum creatinine decreased in 3 cats even resulting in an improvement in IRIS stage, from IRIS stage 3 to 2 in one case. In CKD cats, improvement of clinical signs related to the disease or improvement of renal laboratory parameters even to the extent of an IRIS stage reduction is very unexpected. In particular, for untreated cats and cats in advanced stages of the disease, a worsening over time is expected, due to the progressive pathology of the disease. None of the cats evaluated in this trial received any renal standard of care (in case of renal diet or administration of phosphate binders, the patient had to be stable on such already at screening), confirming the beneficial effect of the oral treatment of 1 mg/kg bodyweight velagliflozin once daily in feline CKD patients. In addition, risk factors for progression of the disease remained stable and within the reference range (e.g. UPC, blood pressure, potassium, phosphorus, etc), suggesting a preventive effect of velagliflozin treatment on disease progression and potential target organ damage.

### Example 4 Exploratory Clinical Field Trial in Client Owned Dogs With CKD

Client owned patients diagnosed with CKD diagnosed via physical examination and biomarker levels are orally treated once daily with velagliflozin 0.05 mg/kg bodyweight. During the study period the body weight, body condition score, biomarker levels (creatinine and SDMA), proteinuria, presence of concomitant conditions and need for further medication are examined on regular basis during the visits at the site by the investigator. The parameters examined are creatinine and SDMA levels, proteinuria, blood pressure and hydration status. Additionally, variables as the delayed onset of renal failure, survival time of the dog patients, IRIS stage progression and renal related mortality and/or morbidity are observed. Study results confirm a beneficial effect on renal function and a delay in progression of the disease.

### REFERENCES

(1) Dekkers and Gansevoort, Nephrol Dial Transplant (2020) 35: i33-i42
(2) EP 3 508 222
(3) Wheeler et al., Diabetes Ther (2020) 11:2757-2774
(4) WO 2014/016381
(5) WO 2017/032799
(6) WO 2021/092341

1. One or more SGLT-2 inhibitors or pharmaceutically acceptable forms thereof for use in a method of prevention and/or treatment of one or more renal diseases in a non-human mammal, preferably a carnivore, more preferably a cat or a dog.

2. The one or more SGLT-2 inhibitors or pharmaceutically acceptable forms thereof for the use according to claim 1, wherein the one or more renal diseases are selected from the group consisting of: renal dysplasia, glomerulopathy, polycystic kidney disease, amyloidosis, tubulo-nephritis/tubulointerstitial nephritis (TIN), acute kidney disease, chronic kidney disease.

3. The one or more SGLT-2 inhibitors or pharmaceutically acceptable forms thereof for the use according to claim 2, wherein the one or more renal diseases are selected from the group consisting of: acute kidney disease, chronic kidney disease.

4. The one or more SGLT-2 inhibitors or pharmaceutically acceptable forms thereof for the use according to claim 3, wherein the one or more renal diseases are selected from the group consisting of: chronic kidney disease.

5. The one or more SGLT-2 inhibitors or pharmaceutically acceptable forms thereof for the use according to any one of claims 1 to 4, wherein the one or more SGLT-2 inhibitors are glucopyranosyl-substituted benzene derivatives.

6. The one or more SGLT-2 inhibitors or pharmaceutically acceptable forms thereof for the use according to any one of claims 1 to 5, wherein the one or more SGLT-2 inhibitors are selected from the group consisting of:
(1) a glucopyranosyl-substituted benzene derivative of the formula (1) 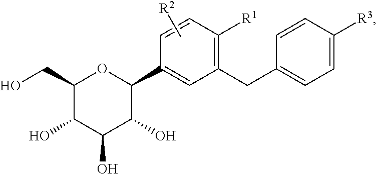
wherein R¹ denotes cyano, Cl or methyl (most preferably cyano);
R² denotes H, methyl, methoxy or hydroxy (most preferably H) and
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoro-methyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetra-hydrofuran-3-yloxy or cyano;
wherein R³ is preferably selected from cyclopropyl, ethyl, ethinyl, ethoxy, (R)-tetrahydrofuran-3-yloxy or (S)-tetrahydrofuran-3-yloxy; and most preferably R³ is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenyl-carbonyl and phenyl-(C₁₋₃-alkyl)-carbonyl;
(2) Velagliflozin, represented by formula (2): 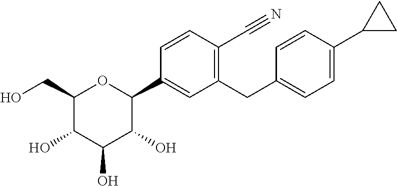 [SMILES: N#Cc1ccc([C@@H]2O[C@H](CO)[C@@H](O)[C@H](O)[C@H]2O)cc1Cc1ccc(C2CC2)cc1]
(3) Dapagliflozin, represented by formula (3): 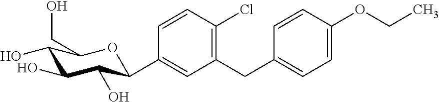
(4) Canagliflozin, represented by formula (4): 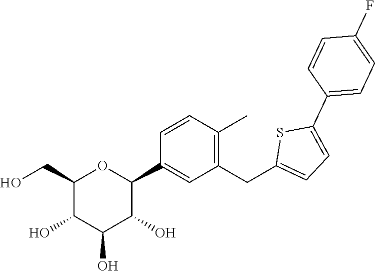 [SMILES: Cc1ccc([C@@H]2O[C@H](CO)[C@@H](O)[C@H](O)[C@H]2O)cc1Cc1ccc(-c2ccc(F)cc2)s1]
(5) Empagliflozin, represented by formula (5): 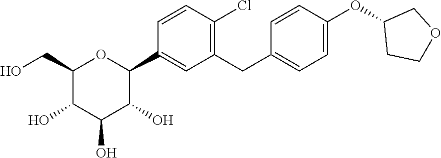 [SMILES: OC[C@H]1O[C@@H](c2ccc(Cl)c(Cc3ccc(O[C@H]4CCOC4)cc3)c2)[C@H](O)[C@@H](O)[C@@H]1O]
(6) Luscogliflozin, represented by formula (6): 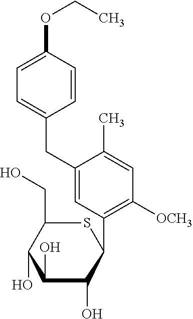
(7) Tofogliflozin, represented by formula (7): 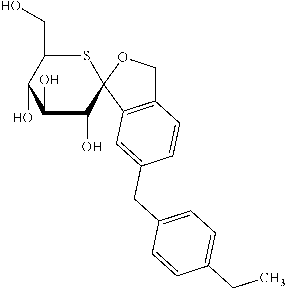
(8) Ipragliflozin, represented by formula (8): 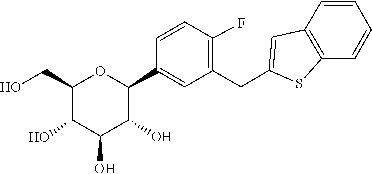 [SMILES: OC[C@H]1O[C@@H](c2ccc(F)c(Cc3cc4ccccc4s3)c2)[C@H](O)[C@@H](O)[C@@H]1O]
(9) Ertugliflozin, represented by formula (9): 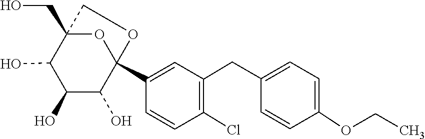 [SMILES: CCOc1ccc(Cc2cc([C@]34OC[C@](CO)(O3)[C@@H](O)[C@H](O)[C@H]4O)ccc2Cl)cc1]
(10) Atigliflozin, represented by formula (10): 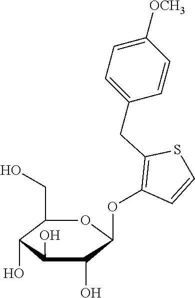
(11) Remogliflozin, represented by formula (11): 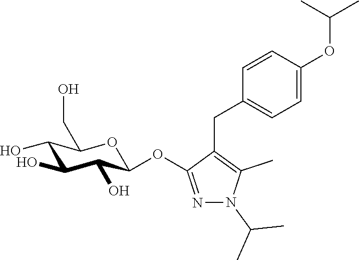
(11A) Remogliflozin etabonate, represented by formula (11A): 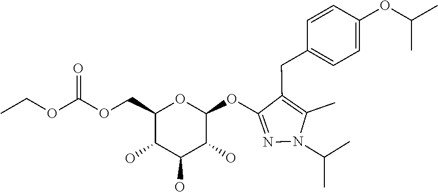 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]
(12) a thiophene derivative of the formnia (12) 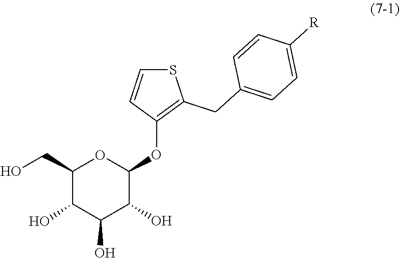
wherein R denotes methoxy or trifluoromethoxy;
(13) 1-(β-D-glucopyranosyl)-4-methyl-3-[5-(4-fluorophenyl)-2-thienylmethyl]benzene, represented by formula (13); 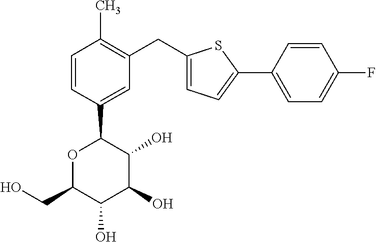 [SMILES: Cc1ccc([C@@H]2O[C@H](CO)[C@@H](O)[C@H](O)[C@H]2O)cc1Cc1ccc(-c2ccc(F)cc2)s1]
(14) a spiroketal derivative of the formula (14): 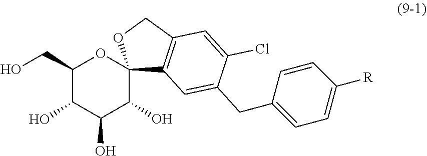
wherein R denotes methoxy, trifluoromethoxy, ethoxy, ethyl, isopropyl or tert. butyl;
(15) a pyrazole-O-glucoside derivative of the formula (15) 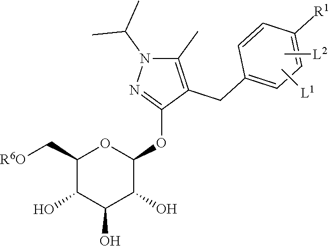
wherein
R¹ denotes C₁₋₃-alkoxy,
L¹, L² independently of each other denote H or F,
R⁶ denotes H, (C₁₋₃-alkyl)carbonyl, (C₁-6-alkyl)oxycarbonyl, phenyloxycarbonyl, benzyloxycarbonyl or benzylcarbonyl;
(16) Sotagliflozin, represented by formula (16): 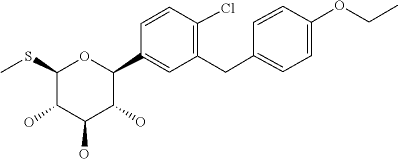 [SMILES: CCOc1ccc(Cc2cc([C@@H]3O[C@H](SC)[C@@H](O)[C@H](O)[C@H]3O)ccc2Cl)cc1]
(17) Sergliflozin, represented by formula (17): 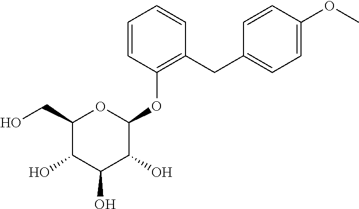 [SMILES: COc1ccc(Cc2ccccc2O[C@@H]2O[C@H](CO)[C@@H](O)[C@H](O)[C@H]2O)cc1]
(18) a compound represented by formula (18): 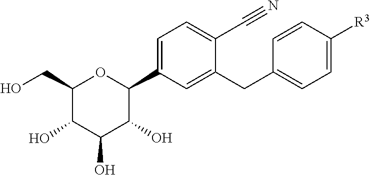
wherein
R³ denotes cyclopropyl, hydrogen, fluorine, chlorine, bromine, iodine, methyl, ethyl, propyl, isopropyl, butyl, sec-butyl, iso-butyl, tert-butyl, 3-methyl-but-1-yl, cyclobutyl, cyclopentyl, cyclohexyl, 1-hydroxy-cyclopropyl, 1-hydroxy-cyclobutyl, 1-hydroxy-cyclopentyl, 1-hydroxy-cyclohexyl, ethinyl, ethoxy, difluoromethyl, trifluoromethyl, pentafluoroethyl, 2-hydroxyl-ethyl, hydroxymethyl, 3-hydroxy-propyl, 2-hydroxy-2-methyl-prop-1-yl, 3-hydroxy-3-methyl-but-1-yl, 1-hydroxy-1-methyl-ethyl, 2,2,2-trifluoro-1-hydroxy-1-methyl-ethyl, 2,2,2-trifluoro-1-hydroxy-1-trifluoromethyl-ethyl, 2-methoxy-ethyl, 2-ethoxy-ethyl, hydroxy, difluoromethyloxy, trifluoromethyloxy, 2-methyloxy-ethyloxy, methylsulfanyl, methylsulfinyl, methlysulfonyl, ethylsulfinyl, ethylsulfonyl, trimethylsilyl, (R)-tetrahydrofuran-3-yloxy or (S)-tetrahydrofuran-3-yloxy or cyano, and wherein R³ is preferably selected from cyclopropyl, ethyl, ethinyl, ethoxy, (R)-tetrahydrofuran-3-yloxy or (S)-tetrahydrofuran-3-yloxy; and R³ most preferably is cyclopropyl,
or a derivative thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl and phenyl-(C₁₋₃-alkyl)-carbonyl;
(19) Bexagliflozin, represented by formula (19): 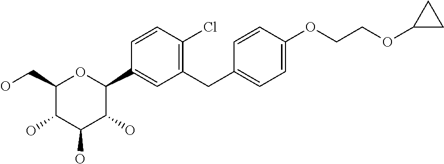 [SMILES: OC[C@H]1O[C@@H](c2ccc(Cl)c(Cc3ccc(OCCOC4CC4)cc3)c2)[C@H](O)[C@@H](O)[C@@H]1O]
(20) Janagliflozin, represented by formula (20): 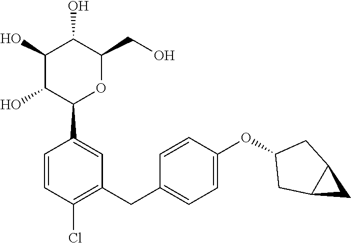 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]
(21) Rongliflozin, represented by formula (21): 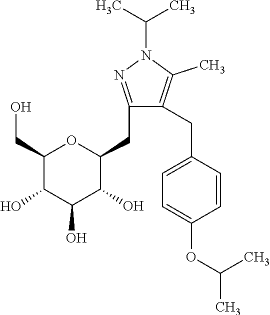 [SMILES: Cc1c(Cc2ccc(OC(C)C)cc2)c(C[C@@H]2O[C@H](CO)[C@@H](O)[C@H](O)[C@H]2O)nn1C(C)C]
(22) Wanpagliflozin.

7. The one or more SGLT-2 inhibitors or pharmaceutically acceptable forms thereof for the use according to any one of claims 1 to 6, wherein the pharmaceutically acceptable form thereof is a crystalline complex between the one or more SGLT2 inhibitors and one or more amino acids, preferably proline, more preferably L-proline; and most preferably is co-crystal of the one or more SGLT2 inhibitors, L-proline and crystalline water.

8. The one or more SGLT-2 inhibitors or pharmaceutically acceptable forms thereof for the use according to any one of claims 1 to 7, wherein the non-human animal, preferably a carnivore, more preferably a cat or a dog, is a non-human animal patient in need of such prevention and/or treatment, preferably a carnivore patient in need of such prevention and/or treatment, and more preferably is a cat patient or a dog patient in need of such prevention and/or treatment, even more preferably a non-diabetic cat patient or a non-diabetic dog patient in need of such prevention and/or treatment.

9. The one or more SGLT-2 inhibitors or pharmaceutically acceptable forms thereof for the use according to any one of claims 1 to 8, wherein the one or more SGLT-2 inhibitors are administered orally, parenterally, intravenously, subcutaneously or intramuscularly, preferably orally.

10. The one or more SGLT-2 inhibitors or pharmaceutically acceptable forms thereof for the use according to any one of claims 1 to 9, wherein the one or more SGLT-2 inhibitors are to be administered at a dose of 0.01 mg/kg bodyweight to 10 mg/kg bodyweight, preferably at a dose of 0.01 mg/kg bodyweight to 5 mg/kg bodyweight, more preferably at a dose of 0.01 mg/kg bodyweight to 4 mg/kg bodyweight, even more preferably at a dose of 0.01 mg/kg bodyweight to 3 mg/kg bodyweight, even more preferably at a dose of 0.01 mg/kg bodyweight to 2 mg/kg bodyweight, even more preferably at a dose of 0.01 mg/kg bodyweight to 1 mg/kg bodyweight, even more preferably at a dose of 0.01 mg/kg bodyweight to 0.5 mg/kg bodyweight, even more preferably at a dose of 0.01 mg/kg bodyweight to 0.3 mg/kg bodyweight, most preferably at a dose of 0.05 mg/kg bodyweight or 1.0 mg/kg bodyweight.

11. The one or more SGLT2 inhibitors or pharmaceutically acceptable forms thereof for the use according to any one of claims 1 to 10, wherein such one or more SGLT2 inhibitors or pharmaceutically acceptable forms thereof is to be administered once per day or twice per day.

12. The one or more SGLT2 inhibitors or pharmaceutically acceptable forms thereof for the use according to any one of claims 1 to 11, wherein the one or more SGLT-2 inhibitors is velagliflozin, which is to be administered as single SGLT-2 inhibitor, preferably orally, more preferably once or twice per day at a dose of 0.01 mg/kg bodyweight to 1 mg/kg bodyweight, even more preferably at a dose of 0.01 mg/kg bodyweight to 0.5 mg/kg bodyweight, even more preferably at a dose of 0.01 mg/kg bodyweight to 0.3 mg/kg bodyweight, most preferably once daily at a dose of 0.05 mg/kg bodyweight or 1.0 mg/kg bodyweight.

13. The one or more SGLT2 inhibitors or pharmaceutically acceptable forms thereof for the use according to claim 12, wherein velagliflozin as single SGLT-2 inhibitor is to be orally administered once daily at a dose of 0.01 mg/kg bodyweight to 1.0 mg/kg bodyweight, preferably at a dose of 0.05 mg/kg bodyweight or 1.0 mg/kg bodyweight.

14. The one or more SGLT2 inhibitors or pharmaceutically acceptable forms thereof for the use according to any one of claims 1 to 13, wherein the one or more SGLT-2 inhibitors are to be administered before, after or concomitantly with administering one or more other active pharmaceutical ingredients, preferably selected from the group consisting of: another SGLT-2 inhibitor or a pharmaceutically acceptable form thereof; one or more ACE inhibitors, such as benazepril, ramipril or enalapril; one or more calcium channel blockers, such as diltiazem or amlodipine; one or more angiotensin receptor blockers, such as telmisartan; one or more calcium-channel sensitizers and/or positive inotropes, such as pimobendan and/or digitalis alkaloids; and/or one or more phosphate binders, such as chitosan.

15. The one or more SGLT2 inhibitors or pharmaceutically acceptable forms thereof for the use according to any one of claims 1 to 14, wherein the preventive and/or therapeutic effect is characterized by one or more of the following clinical and/or biochemical parameters:
improved renal efficiency, characterized by a reduction of proteinuria—as well as a reduction and/or stabilization of serum SDMA and/or serum creatinine;
increase of the production of ketone bodies in the liver, characterized by increased plasma levels of 3-hydroxybutyric acid and/or the corresponding acylcarnitines i.e. hydroxybutyrylcarnitine and increased plasma levels of one or more of the branched-chain amino acids (e.g. valine, leucine and isoleucine);
improved blood pressure;
improved hydration status;
delayed onset of renal failure, preferably at least by 1, 2, 3, 4, 5, 6, 7, 8, 9, 10, 11, 12 or more months, or delayed and/or stopped progression of the one or more renal diseases, in particular chronic kidney disease, and/or improvement of the classification stage of the one or more renal diseases, in particular CKD (e.g. from stage III to stage II);
longer survival time of the non-human mammal patient, preferably at least by 1, 2, 3, 4, 5, 6, 7, 8, 9, 10, 11, 12 or more months and/or lower renal related mortality and/or morbidity;
improved clinical signs, such as reduced polydipsia, polyuria, vomiting and/or lethargy;
higher quality of life.

16. A pharmaceutical composition comprising one or more SGLT2 inhibitors or pharmaceutically acceptable forms thereof according to any one of claims 1 to 15 for use according to any one of claims 1 to 15.
